(19) United States
(12) Patent Application Publication
(10) Pub. No.: US 2021/0100859 A1
(43) Pub. Date: Apr. 8, 2021

(54) HERPES SIMPLEX VIRUS (HSV) ANTICANCER THERAPIES

(71) Applicants: THE BOARD OF REGENTS OF THE UNIVERSITY OF TEXAS SYSTEM, Austin, TX (US); THE OHIO STATE UNIVERSITY, Columbus, OH (US)

(72) Inventors: Balveen KAUR, Houston, TX (US); Selvendiran KARUPPAIYAH, Columbus, OH (US); Bangxing HONG, Houston, TX (US)

(21) Appl. No.: 17/066,162

(22) Filed: Oct. 8, 2020

Related U.S. Application Data
(60) Provisional application No. 62/912,476, filed on Oct. 8, 2019.

(51) Int. Cl. A61K 35/768; A61P 35/00; A61K 33/243; C07K 16/28

(52) U.S. Cl. CPC A61K 35/768; C07K 16/2818; A61K 33/243; A61P 35/00

(57) ABSTRACT

The present disclosure provides methods for treating cancer comprising administering an oncolytic virus, such as an oncolytic herpes simplex virus, in conjunction with a DNA damaging agent (e.g., cisplatin).

This application claims the benefit of U.S. Provisional Application Ser. No. 62/912,476, filed Oct. 8, 2019, the entirety of which is incorporated herein by reference.

The sequence listing that is contained in the file named “UTSHP0360US_ST25.txt”, which is 6 KB (as measured in Microsoft Windows) and was created on Oct. 8, 2020, is filed herewith by electronic submission and is incorporated by reference herein.

### BACKGROUND

### 1. Field

The present disclosure relates generally to the field of biology and medicine. More particularly, it concerns methods for the treatment of cancer, such as ovarian cancer.

### 2. Description of Related Art

Despite the approval of therapies such as avastin and PARP inhibitors, ovarian cancer has the highest case-fatality ratio of any gynecologic cancer (SEER, 2015). First-line chemotherapy for the treatment of OC using platinum-based drugs (CP, carboplatin) yields a response rate of >80%; however, over 50% of patients eventually suffer from recurrence (Davis et al., 2014; Holmes, 2015; Agarwal and Kaye, 2003), and recurrent OC is almost always resistant to platinum based therapy. Patients with platinum-resistant ovarian cancer have a median survival of 6 months, and only 27% live longer than 12 months (Chan et al., 2008). Thus, strategies to treat cisplatin resistant ovarian cancer treatment are urgently needed.

Oncolytic viruses are live viruses designed to infect and specifically destroy cancer cells. Amgen is currently marketing an FDA approved oncolytic HSV for treatment of metastatic melanoma, and several other ongoing trials with numerous different OV are being tested for efficacy in ovarian cancer patients (NCT03663712 (HSV-1) NCT02759588 (GL-ONC1); NCT02364713 (Measles); NCT01199263 (Reovirus); NCT03225989 (Adenovirus); NCT03120624 (VSV)). However, the impact of oHSV therapy on cisplatin resistance for OC is not known.

The impact of Cisplatin and oHSV on therapeutic efficacy appears to depend on tumor type and oncolytic virus used. Therapeutic efficacy of combining oHSV and cisplatin has been reported to be both beneficial and antagonistic. For example, treatment of human bladder carcinoma with cisplatin and oHSV was found to be antagonistic (Simpson et al., 2012), but increased apoptotic cell death was observed for the combination treated for pancreatic cancer cells in vitro (Kasuya et al., 2007). Similarly, while cisplatin has been shown to function as an anti-herpetic (HSV-2) agent in normal cells (Snyder et al., 1987), Cisplatin induced GADD34 expression in malignant peripheral nerve sheath tumor cells is also thought to increase OV replication and lysis (Adusumilli et al., 2006). To date there has been no report that has investigated the impact of oHSV therapy and cisplatin in ovarian cancers. The efficacy of combining cisplatin and oHSV against orthotopic and peritoneal ovarian cancers has also not been described. Thus, there is an unmet need for improved therapies in these cancers.

### SUMMARY

In some embodiments, the present disclosure provides methods of treating a subject having a cancer comprising administering an effective amount of an oncolytic virus to the subject in conjunction with at least a first DNA damaging agent. In some aspects, the DNA damaging agent is a DNA-damaging chemotherapeutic agent. In some aspects, the DNA damaging agent is a platinum-based DNA damaging agent. In some aspects, the platinum-based DNA damaging agent comprises cisplatin, oxaliplatin and/or carboplatin. In some aspects, the platinum-based DNA damaging agent comprises cisplatin. In some aspects, the oncolytic virus is an oncolytic herpes simplex virus (oHSV). In some aspects, the oHSV is an HSV-1 strain. In some aspects, the oHSV is a Vstat120-expressing oHSV, such as Rapid Antiangiogenesis Mediated By Oncolytic Virus (RAMBO). In particular aspects, the subject is administered a Vstat120-expressing oHSV in combination with a platinum-based DNA damaging agent, such as cisplatin, oxaliplatin, and/or carboplatin.

In some aspects, the amount of the oncolytic virus is effective to stimulate an innate and/or adaptive immune response in the subject. In some aspects, the amount of the oncolytic virus is effective to stimulate an anti-tumor T-cell response in the subject. In some aspects, the amount of the oncolytic virus is effective to stimulate PD1-PDL1 signaling in T-cells and/or myeloid cells of the subject. In some aspects, the oncolytic virus is administered after the DNA damaging agent. In some aspects, the oncolytic virus is administered before or essentially simultaneously with the DNA damaging agent. In some aspects, the oncolytic virus is administered within 1 day of the DNA damaging agent. In some aspects, the oncolytic virus is administered within 3 hours of the DNA damaging agent. In some aspects, the oncolytic virus is administered to the subject 2, 3, 4 or 5 times.

In some aspects, the subject has a solid tumor. In some aspects, the cancer is ovarian cancer. In some aspects, the cancer is orthotopic or peritoneal ovarian cancer. In some aspects, the cancer is metastatic cancer. In some aspects, the cancer is resistant to at least one anti-cancer therapy. In some aspects, the cancer is resistant to a DNA-damaging chemotherapeutic agent. In some aspects, the cancer is a platinum resistant cancer. In some aspects, the cancer is a cisplatin resistant cancer. In some aspects, the cancer is a cisplatin resistant ovarian cancer. In some aspects, the oncolytic virus is administered systemically. In some aspects, the oncolytic virus is administered intraperitoneally. In some aspects, the oncolytic virus is administered locally. In some aspects, the oncolytic virus is administered intratumorally.

In some aspects, the methods further comprise administering a further anti-cancer therapy. In some aspects, the further anti-cancer therapy is selected from the group consisting of a chemotherapy, a radiotherapy, an immunotherapy, or a surgery. In some aspects, the immunotherapy comprises at least one immune checkpoint inhibitor. In some aspects, the immune checkpoint inhibitor inhibits the PD1-PDL1 pathway. In some aspects, the immune checkpoint inhibitor is an anti-PD1 or anti-CTLA-4 monoclonal antibody. In some aspects, the immunotherapy is a combination of immune checkpoint inhibitors.

In another embodiment, the present disclosure provides pharmaceutical compositions comprising an oncolytic virus and a DNA damaging chemotherapeutic agent.

In still another embodiment, the present disclosure provides compositions for use in treating a subject having a platinum resistant cancer, the composition comprising an effective amount of an oncolytic virus. In some aspects, the oncolytic virus is an oncolytic herpes simplex virus (oHSV). In some aspects, the compositions are for use in treating a subject having a platinum resistant ovarian cancer. In some aspects, the compositions are for use in treating a subject having a cisplatin resistant ovarian cancer.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

As used herein in the specification and claims, the term “RAMBO” (Rapid Antiangiogenesis Mediated By Oncolytic Virus) is an oncolytic HSV-1 (oHSV) which expresses a potent anti-angiogenic gene Vasculostatin (Vstat120) and is described in U.S. Pat. No. 8,450,106 and also described in Hardcastle et al. (2010), both incorporated by reference herein in their entirety.

Other objects, features and advantages of the present disclosure will become apparent from the following detailed description. It should be understood, however, that the detailed description and the specific examples, while indicating certain embodiments of the present disclosure, are given by way of illustration only, since various changes and modifications within the spirit and scope of the present disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1J: RAMBO increases cisplatin accumulation in cisplatin-resistant ovarian cancer cells. (FIG. 1A) Representative histogram (left) and quantification (right) of percentage of GFP⁺ infected cisplatin resistant ovarian cancer cells. TR127 cells infected with RAMBO (MOI=0.1) for 1 hr and then treated with CP (2 μM) for 16 hr. Data shown are mean percentage of GFP⁺ RAMBO infected TR127 cells (±s.d) analyzed by flow cytometry (n=3/group). (FIG. 1B) Quantification of intracellular CP retained inside platin resistant OC cells treated with/without RAMBO. OVCAR8, TR127 and TR182 were treated with/without RAMBO (MOI=0.1) for one hour and then incubated with 2.5 μM Texas-red labeled CP. Twenty-four hours post-infection CP accumulation in cells was analyzed by flow cytometry. Data shown are mean fluorescence intensity (MFI) of Texas-red labeled CP (±s.d., n=3/group). (FIG. 1C) Confocal immuno-fluorescent images of TR127 cells treated with RAMBO (MOI=0.5) (bottom) or untreated (top) for 1 hr and then treated with 2.5 μM Texas-red labeled CP (red) for 24 hr. Cells were stained with DAPI and F-actin to image nuclei (blue) and F-actin (white) respectively. Images on the right are magnifications of the inset on the left. (FIG. 1D) Gene Ontology terms and KEGG pathway enrichment analysis of differentially expressed genes in RAMBO infected versus uninfected OVTOKO cells. Pathways significantly changed in infected cells are shown color coded for fold change. For a pathway, the size and color represent the number of down-regulated genes in that pathway and the False Discovery Rate (FDR), respectively. Briefly, RNA harvested from OVTOKO cells treated with RAMBO (MOI=1) for 16 hr was analyzed by RNAseq. The value shown is the average of three samples for each group. (FIGS. 1E-F) Heatmaps showing changes in gene expression within each panel of the Gene Ontology terms: extracellular exosome (FIG. 1E) and lysosome GO pathways (FIG. 1G). (FIG. 1G) Confocal immuno-fluorescent images of OVTOKO cells treated with Texas-red labeled CP and stained with lysotracker (white) and DAPI (blue). OVTOKO cells were plated in poly-lysine coated chamber slides and infected with RAMBO (MOI=1) for 16 hr and then treated with Texas-red labeled CP (2 μM) for another 4 hr. Infected cells become GFP+ve (bottom). (FIG. 1H) Western blot analysis of efflux proteins ATP11B and ATP7B expression in TR127 and TR182 cells treated with RAMBO and/or CP. (FIG. 1I) Quantification of Texas-red labeled CP accumulation in TR127 cells knocked down for either ATP7B, ATP11B, or both was analyzed by flow cytometry (n=3/group). (FIG. 1J) Quantification of Texas-red labeled CP accumulation in OVTOKO cells overexpressing (OE) both ATP11B and ATP7B, or control (Ctl). Cells were analyzed by flow cytometry. Error bars are s.d., *p value<0.05, CP=Cisplatin, R=RAMBO.

FIGS. 2A-2G: Effect of RAMBO on DNA damage and STING activation in cisplatin resistant ovarian cancer cells. (FIG. 2A) Representative histograms from flow cytometry analysis of cisplatin-specific DNA adducts in CP treated TR127 and TR182 cells also treated either with RAMBO (solid line) or without (dotted line). Isotype control is shaded in grey. Right panel shows the quantification of cisplatin-specific DNA adducts in TR127 and TR182 cells treated with/without RAMBO (MOI=0.1) and with/without CP (2 μM) for 16 hr. (shaded=isotype, dash line=CP, solid line=RAMBO+CP, n=3/group). (FIG. 2B) Western blot analysis of p-Chk2 and p-H2X in TR182 (left) and TR127 (right) cells treated with RAMBO (MOI=0.1) and/or CP (2 μM) and harvested 0, 1, 16, or 30 hours post treatment. (FIG. 2C) Immunofluorescence staining of micronuclei (extranuclear γH2AX staining) in OVTOKO control or cells treated with RAMBO and/or CP. p-H2AX (red) was used as a marker for damaged DNA double strand breaks, tumor cell nuclei is stained with DAPI (blue). p-H2AX staining outside the nucleus marks micronuclei containing damaged DNA. (FIG. 2D) Heatmaps of differentially expressed genes in the different signaling pathways derived from an RNAseq analysis of OVTOKO cells treated with/without RAMBO and with/without CP. (FIG. 2E) GSEA plot for cGAS-STING signaling and type I interferon signaling in OVTOKO cells treated with RAMBO and CP relative to untreated cells (top), or CP alone (bottom) treated cells. (FIG. 2F) Quantification of cGAMP in the culture supernatant of OVTOKO cells treated with RAMBO (MOI=1) and/or CP (5 μM) for 16 hr as measured by ELISA (n=3/group)). (FIG. 2G) Quantification of IFNβ mRNA in OVTOKO cells treated with RAMBO (MOI=1) and/or CP (5 μM) for 16 hr analyzed by qRT-PCR (n=3/group). Error bars are s.d, *p value<0.05, M=Mock, CP=Cisplatin, R=RAMBO, RCP=RAMBO+CP.

FIGS. 3A-3F: Combination RAMBO with cisplatin treatment increases innate immunity. (FIGS. 3A-B) Quantification of viable TR127 and TR182 cells treated with RAMBO and/or CP with PBMCs (black bars) or without (grey) overlaid. Cell tracker labelled TR127 cells (FIG. 3A) or TR182 cell (FIG. 3B) were infected with RAMBO (MOI 0.04) for 1 hr and then treated with or without CP (5 μM) and overlaid with or without PBMCs (Effector: Target=10:1) for another 96 hrs. Data shown are mean viable tumor cells (annexin and PI double negative)±s.d quantified by flow cytometry (n=3/group). (FIG. 3CA) Quantification of IFNγ secretion in PBMCs co-cultured with TR127 or TR182 cells treated with RAMBO and/or CP. Data shown are mean IFNγ±s.d released into conditioned medium (CM) (n=3/group). (FIG. 3D) Quantification of gene expression of NKG2D ligands: ULBP1, ULBP2, ULBP3, MICA, and MICB relative to GAPDH by qRT-PCR from TR182 cells treated with RAMBO (MOI=0.2) and/or CP (2 μM) for 16 hours (n=3/group). (FIG. 3E) Representative dot blots and quantification of NKG2D expression (activation marker) on CD56⁺ NK cells following co-cultured with OVTOKO cells by flow cytometry. OVTOKO cells were treated with RAMBO (MOI=0.5) and/or CP (5 μM) for 16 hrs and then overlaid with NK cells (Effector:Target=2:1) isolated from human donor-derived PBMCs for 48 hr. (FIG. 3F) Mean IFNγ secretion in the supernatants from OVTOKO and NK cell co-cultures quantified by ELISA (n=3/group). Error bars are s.d, *p value<0.05, CP=Cisplatin.

FIGS. 4A-4H: Combination of RAMBO with cisplatin treatment increases anti-tumor activity in TR182-Luc/NSG model. (FIG. 4A) Treatment schematic TR182-luc xenograft establishment and therapy in NSG mice. 6-week old female NSG mice were inoculated with 5×10⁵ TR182-Luc-GFP tumor cells intraperitoneally. 5 days later tumor-bearing mice were treated with 5×10⁵ pfu RAMBO with or without CP (2 mg/Kg) given on the indicated days. (FIGS. 4B-C) IVIS imaging and quantification TR182-Luc tumors in NSG mice after treatment with RAMBO and/or CP. Tumor growth of individual mice was monitored by IVIS imaging on day-1, day 18 and day 25 after RAMBO treatment (n=9/group, dpi=days post infection). (FIGS. 4D-E) Quantification of tumor nodules (FIG. 4D) and tumor weights (FIG. 4E) in TR182-Luc implanted mice twenty-eight days following RAMBO and/or CP treatment. (FIG. 4F) Representative dot plots and quantification of GFP⁺ TR182 tumor cells and CD45⁺ leukocytes in tumor nodules harvested from TR182-Luc bearing mice twenty-eight days following RAMBO and/or CP treatment. Data shown are mean percentage of GFP⁺ TR182-Luc cells±s.d. (n=3/group). (FIG. 4G) H&E and Ki67 IHC staining of tumor nodules harvested from TR182-Luc-bearing mice 28 days following RAMBO and/or CP treatment. (FIG. 4H) Quantification of tumor associated macrophage phenotype markers CD206 and CD86 expressed in tumor nodules harvested from TR182-Luc bearing mice 28 days following RAMBO and/or CP treatment. Tumor nodules were harvested and single cell suspension were prepared for flow cytometry analysis of % CD86⁺ and % CD206⁺ cells gated on F4/80 (n=3). Error bars denote s.d, *p value<0.05, **p value<0.01, Mock=7/group, CP=6/group, RAMB 0=9/group, RAMBO+CP=9/group, CP=Cisplatin.

FIGS. 5A-5D: Combination of RAMBO with cisplatin treatment increases anti-tumor activity in ID8-Luc C57/BL6 model. (FIG. 5A) Treatment schedule of C57BL/6 mice bearing ID8-Luc-GFP intraperitoneal metastatic tumors. 5×10⁶ ID8-Luc-GFP tumor cells were implanted into 6-week old female C57BL/6 mice. 15 days later tumor-bearing mice were treated with or without 5×10⁵ pfu RAMBO (day 15) with or without CP (2 mg/Kg, at the indicated time points). (FIGS. 5B-C) IVIS imaging and quantification of luciferase in ID8-Luc-GFP tumors grown in C57BL/6 mice following RAMBO and/or CP treatment. Tumor growth was monitored by IVIS imaging on day −1, day 23, and day 49 following RAMBO and CP treatments (n=10/group, dpi=days post infection). (FIG. 5D) Quantification of volume of ascites in ID8-luc-GFP-bearing C57BL6 mice after RAMBO and/or CP treatment on day 75 following RAMBO treatment (n=5/group). Error bars are s.d, *p value<0.05, CP=cisplatin.

FIGS. 6A-6H: Combination of RAMBO with cisplatin treatment activates an anti-tumor immune response. (FIG. 6A) Percent quantification of dendritic cells expressing CD86 and MHCII in splenocytes of mice ted with RAMBO and/or CP. Three days following RAMBO treatment of ID8-Luc-GFP tumor-bearing mice, splenocytes were harvested and analyzed for % CD86⁺, and % MHCII⁺ CD11c⁺ dendritic cells (n=3/group). (FIG. 6B) Quantification of cytosolic DNA and mitochondrial DNA in human donor PBMC-derived dendritic cells co-cultured with OVTOKO cells treated with RAMBO and/or CP. Cells were co-cultured for 48 hrs, cytosolic DNA and mtDNA from dendritic cells was quantified by qRT-PCR (n=3/group). c: Gene expression quantification by qRT-PCR of cGAS from OVTOKO cells (left) in bottom and dendritic cells derived from the transwell experiment described in (FIG. 6B), (n=3/group). (FIG. 6D) Quantification of IFNβ mRNA from human PBMC-derived dendritic cells knocked down for STING (siSTING) and co-cultured with OVTOKO cells treated with RAMBO and/or CP compared to control. IFNβ mRNA was quantified by qRT-PCR (n=3/group). (FIG. 6E) Quantification of % CD44⁺CD8⁺ and CD44⁺CD4⁺ T cells co-cultured human donor PBMC-derived dendritic cells and OVTOKO cells treated with RAMBO and/or CP. T cell activation was measured by flow cytometry. (FIG. 6F) IFNγ secretion in the supernatant of co-cultures from RAMBO and/or CP treated OVTOKO cells with human PBMC-derived dendritic cells and purified T cells was quantified by ELIS, (n=3/group). (FIGS. 6G-H) Dot plots and quantification of PD1 expression in splenic T cells from ID8-Luc-GFP tumor-bearing mice. Three days following RAMBO treatment, splenocytes were harvested and analyzed for % PD1 expression on CD4⁺ and CD8⁺ T cells by flow cytometry (n=3). Error bars denote s.d, *p value<0.05, **p value<0.001, NS indicated no significant difference, CP=Cisplatin

FIGS. 7A-7H: PD1 blockade increases anti-tumor response in murine metastatic ovarian cancer ID8-Luc model treated with RAMBO and cisplatin. (FIG. 7A) Representative histogram and quantification of PD-L1 in ovarian cancer cells treated with RAMBO and/or CP. TR127 cells were treated with RAMBO (MOI=0.1) and/or CP (5 μM) for 24 hr. PD-L1 expression was analyzed by flow cytometry (left). OVTOKO were treated with RAMBO (MOI=0.5) and/or CP (5 μM) for 24 hr. PD-L1 expression was analyzed by qRT-PCR (right), (n=3). (FIG. 7B) Schematic of establishment and treatment of ID8-Luc-GFP tumor model in C57BL/6 mice treated with RAMBO and CP, with or without an anti-PD-1 antibody. 5×10⁶ ID8-Luc-GFP tumor cells were inoculated intraperitoneally into 6-week old female C57BL/6 mice. 15 days later, tumor-bearing mice were treated with 5×10⁵ pfu RAMBO and CP (2 mg/kg) with or without anti-PD1 antibody (100 μg/mouse). (FIGS. 7C-D) IVIS imaging and quantification of ID8-Luc-GFP tumor growth in C57BL/6 mice on day −1, day 12, and day 65 following treatment (n=10). (FIGS. 7E-H) Flow cytometry analysis of splenocytes from mice treated with RAMBO and CP with or without anti-PD-1 blocking antibody. ((FIG. 7E) Quantification of NK cell activation markers (NK1.1 and NKG2D). (FIG. 7E) Quantification of % PD-1⁺CD4⁺ and CD8⁺ T cells. (FIG. 7G) Quantification of % Tim-3⁺ CD4⁺ and CD8⁺ T cells, and (FIG. 7H) Quantification of % CD62L⁻ CD4⁺ and CD8⁺ of T cells, (n=3). Error bars are s.d, *p value<0.05, ns indicated no significant difference, CP=Cisplatin.

FIG. 8: ATP11B and ATP7B silencing in OC cells. Gene expression quantification of ATP11B and ATP7B knockdown in TR127 cells. TR127 cells were transfected with 100 nM ATP11B siRNA, ATP7B siRNA, or both for 48 hr. ATP11B and ATP7B mRNA was measured by qRT-PCR (n=3). Error bars are s.d, **p value<0.01, CP=Cisplatin, OC=Ovarian Cancer.

FIG. 9: Combination RAMBO and CP therapy reduces viability of OC cells. Quantification of murine OC cell lysis following treatment with RAMBO and/or CP. Murine ID8 OC cells were infected with RAMBO (MOI=0.5) for 1 hr and then treated with or without CP (10 μM) for another 48 hr. Tumor cell killing activity was analyzed by flow cytometry using Annexin-V/PI staining. Live tumor cells are indicated as Annexin-V₋/PI₋ population (n=3). Error bars are s.d, *p value<0.05, CP=Cisplatin, OC=Ovarian cancer.

FIGS. 10A-10B: Combination RAMBO and CP increase NK cell-mediated OC cell lysis. Quantification of NK cell-mediated OC cell lysis following treatment with RAMBO and/or CP. OVTOKO cells treated with RAMBO (MOI=1) and/or CP (5 μM) were co-cultured with purified NK cells (Effector:Target=2:1) derived from human PBMCs for 96 hr. Tumor cell lysis was quantified by flow cytometry analysis of Annexin V/PI staining of CD56₋ tumor cells. (FIG. 10A) Dot plots indicating the ratio of CD56₊ NK cells and CD56₋ tumor cells in the co-culture. (FIG. 10B) Histogram showing the absolute number of live CD56-Annexin-V₋PI₋ tumor cells in the coculture (n=3). Error bars are s.d, **p value<0.01, CP=Cisplatin, OC=Ovarian cancer.

FIGS. 11A-11B: Combination of RAMBO with cisplatin treatment activates cGAS-STING signaling and increases PD-L1 expression in vivo. TR182 tumor-bearing mice were treated with PBS, CP, RAMBO or RAMBO+CP. 24 hrs after treatment, 20 mg of tumor tissue was prepared for RNA extraction. cGAS-STING signaling activation molecules (FIG. 11A) and NK2GD ligands (FIG. 11B) was quantified by qRT-PCR. n=3/group. (FIG. 11B) Quantification of PD-L1 expression in cisplatin resistant OC cells treated with RAMBO and/or CP in vivo. TR182-Luc-GFP metastatic OC model was established and treated with RAMBO and/or CP in NSG mice as indicated in FIG. 4. PD-L1 expression on TR182-Luc-GFP cells was analyzed by flow cytometry at day 28 following RAMBO treatment (n=3). Error bars are s.d, *p value<0.05, **p value<0.01, CP=Cisplatin, OC=Ovarian cancer.

FIG. 12: Combination of RAMBO with cisplatin treatment activates an antitumor immune response. Dot plots of PD1 expression in splenic T cells from ID8-Luc-GFP tumor-bearing mice. Three days following RAMBO treatment, splenocytes were harvested and analyzed for % PD1 expression on CD4⁺ and CD8⁺ T cells by flow cytometry.

FIG. 13: PD-1 blockade decreases FoxP3 regulatory T cells in ID8-Luc C57BL/6 mice treated with RAMBO and CP. Quantification of FoxP3⁺ T_{regs} harvested from splenocytes of ID8-Luc-GFP bearing mice treated with or without an anti-PD-1 antibody, and with or without RAMBO and CP in vivo. The ID8-Luc tumor model was established and treated as indicated in FIG. 7. Flow cytometry analysis of CD4⁺Foxp3⁺ and CD8⁺Foxp3⁺ T cells from the spleen of ID8-Luc-GFP-bearing mice 35 days after RAMBO treatment (n=3). Error bars are s.d, *p value<0.05, ns indicated no significant difference, CP=Cisplatin.

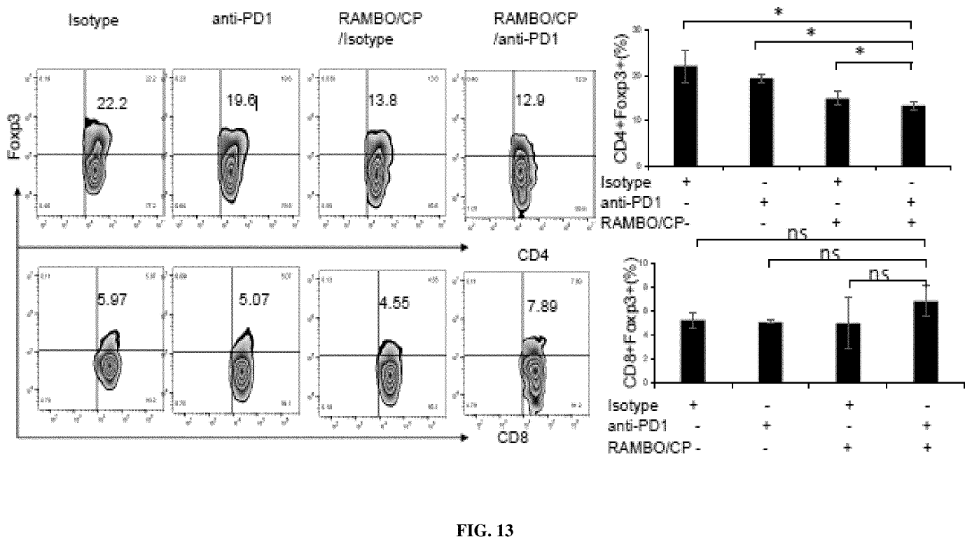

FIG. 14: Western blot analysis of cisplatin resistance genes in primary ovarian cancer TR127 and TR182 cells treated with RAMBO and cisplatin. Primary ovarian cancer cells TR127 and TR182 were treated with RAMBO at M01=0.2 and cisplatin (2 μM) for 48 hrs. Cisplatin resistance molecules DGKA, Foxo3A and HoxD8 expression were analyzed by western blot analysis. Results showed that DGKA and HoxD8 levels were unchanged compared to untreated cells and only Foxo3a showed a slight reduction after RAMBO treatment.

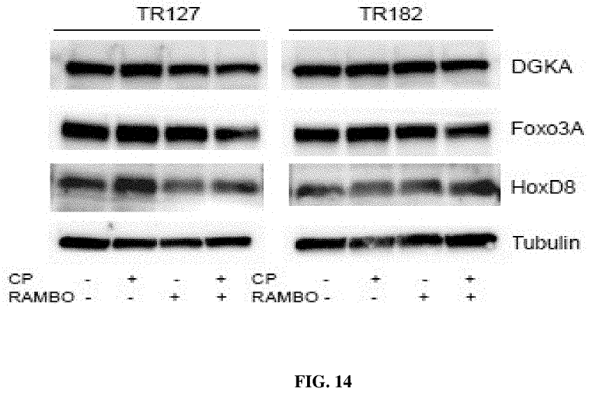

### DESCRIPTION OF ILLUSTRATIVE EMBODIMENTS

### I. The Present Embodiments

Despite the approval of novel therapies such as avastin and PARP inhibitors, ovarian cancer has the highest case-fatality ratio of any gynecologic cancer. First-line chemotherapy for the treatment of ovarian cancer using platinum-based drugs eventually suffer from recurrence, and recurrent ovarian cancer is always resistant to platinum-based chemotherapy. Oncolytic viruses are live viruses designed to infect and specifically destroy cancer cells. Amgen is currently marketing an FDA approved oncolytic HSV for treatment of metastatic melanoma, and several other ongoing trials with numerous different OV are being tested for efficacy in ovarian cancer patients (NCT03663712 (HSV-1) NCT02759588 (GL-ONC1); NCT02364713 (Measles); NCT01199263 (Reovirus); NCT03225989 (Adenovirus); NCT03120624 (VSV)).

In this disclosure, the impact of oncolytic HSV therapy on cisplatin sensitivity was determined in cisplatin resistant ovarian cancer cells in vitro and in vivo metastasis models. These findings uncovered modulation of cisplatin efflux mechanisms by oHSV infection that resulted in degradation of ATP11B and ATP7B, and increased cisplatin accumulation in ovarian cancer cells. This also correlated with increased DNA damage, resulting in induction of micronuclei and the consequential activation of cGAS-STING pathway leading to interferon signaling. This combination resulted in the induction of anti-tumor innate and adaptive immune responses. The induction of checkpoint feedback upregulation indicates it can be used in combination with checkpoint inhibitor therapy for cisplatin resistant cancer cells and in particular ovarian cancer cell treatment. Together this therapy resulted in a strong anti-tumor immune response and increased anti-tumor efficacy in mice treated with the combination.

Specifically, therapeutic efficacy of the combination was assessed in platin resistant human and murine OC peritoneal metastatic mouse models (n=9-10/group). RNA sequencing along with flow cytometry of splenocytes from treated mice was employed to examine the effect of anti-tumor immune response (n=3/group). Anti-PD-1 antibody was performed to evaluate impact on checkpoint inhibition in vivo. Gene ontology pathway analysis uncovered disruption of cellular extracellular vesicle (EV) related pathways in infected cells (FDR=2.97E-57). Reduced expression of transporters expressed on EV implicated in CP efflux was also identified. Increased cisplatin retention led to increased cisplatin-DNA adducts, which resulted in micronuclei and the subsequent activation of cGAS-STING pathway with a significant activation of innate immune cells and translated to an increase in anti-tumor immunity and efficacy. In mice bearing platin resistant OC, a feed-back induction of PD-L1 was also observed on tumor cells, which sensitized the combination treated mice to anti-PD-1 immune checkpoint therapy. This is the first report that oncolytic viruses, such as, but not limited to, HSV can induce cisplatin retention in infected cells. The resulting damaged DNA was then expelled from cells as micronuclei which resulted in induction of inflammatory responses and education of anti-tumor immunity. The combination therapy also created an environment that sensitized tumors to immune checkpoint therapy.

In some embodiments, co-treatment with that oncolytic viruses, such as, but not limited to, oHSV in combination with cisplatin sensitizes ovarian cancer cells to immunotherapy. Oncolytic viruses, such as, but not limited to oHSV infection, may significantly increases cisplatin accumulation in cisplatin-resistant ovarian cancer cells. Oncolytic viruses, such as, but not limited to oHSV infection induces DNA damage response when used in combination with cisplatin in treatment of cisplatin-resistant ovarian cancer cells. Combining oHSV with cisplatin significantly induced cGAS-STING-ISGs signaling and activated the anti-tumor innate and adaptive immune response. Combination of oHSV with cisplatin treatment increased anti-tumor efficacy in TR182-Luc NSG model. Combination of oHSV with cisplatin treatment increased anti-tumor activity in ID8-Luc/C57BL6 model. PD1 blockade further increased oHSV/Cisplatin anti-tumor activity in ID8-Luc/C57BL6 model.

In some embodiments, methods for co-administration of cisplatin and oncolytic viruses, such as, but not limited to oHSV therapies against orthotopic and peritoneal ovarian cancers are provided. In some embodiments, oncolytic HSV therapy enhanced cisplatin sensitivity in cisplatin resistant ovarian cancer cells in in vitro and in vivo metastasis models. In some embodiments, oHSV infection modulates cisplatin efflux mechanisms and results in increased cisplatin accumulation in ovarian cancer cells. In some embodiments, co-administration of cisplatin and oHSV therapies result in modulation of cisplatin efflux mechanisms by oncolytic HSV infection that results in degradation of ATP11B and ATP7B, and the induction of checkpoint feedback upregulation indicating it can be used as neoadjuvant therapy and checkpoint inhibitor therapy for cisplatin resistant ovarian cancer treatment. Thus, co-administration of cisplatin and oHSV resulted in a strong anti-tumor immune response and increased anti-tumor efficacy.

In some embodiments, co-administration of cisplatin and oncolytic viruses such as, but not limited to oHSV can increase tumor DNA damage and in the induction of anti-tumor innate and adaptive immune responses. The treatment resulted in the induction of checkpoint upregulation indicating that checkpoint inhibitors can be used as combination therapy for cisplatin resistant ovarian cancer treatment. Thus, co-administration of cisplatin and the oncolytic virus, oHSV resulted in a strong anti-tumor immune response and increased anti-tumor efficacy.

In some embodiments, co-administration of cisplatin and oHSV therapies result in a reversal or reduction in cisplatin resistance by tumor cells. Recurrent and metastatic ovarian cancer develop different mechanisms of cisplatin resistance. Cisplatin import and export was modified by the oncolytic virus infection and increased sensitivity to cisplatin.

In some embodiments, co-administration of cisplatin and oHSV therapies resulted in an increase in DNA damage response, cGAS-STING activation in cancer radiotherapy or chemotherapy. Radiotherapy and chemotherapy not only directly kill tumor cells, but also induce DNA damage, which activates cGAS-STING pathway in tumor cells and downstream interferon signaling. Interferon signaling could activate both innate and adaptive immune response. The results indicate that oHSV/Cisplatin could not only activate cGAS-STING-interferon signaling and thus activate both innate and adaptive immune response, but also induce PD1-PDL1 signaling including upregulation of PDL1 expression in tumor cells and PD1 expression in both T cells and myeloid cells, cGAS-STING interferon signaling is impaired tumor cells thus making tumor cells, but not healthy cells, more easily infected by oncolytic virus.

In some embodiments, co-administration of cisplatin and oncolytic viruses, such as, but not limited to oHSV therapies may activate interferon signaling. Interferon signaling also induces feedback inhibition such as PD1-PDL1 activation. The upregulation of PD1-PDL1 signaling was demonstrated in in immunodeficient NSG mice targeting myeloid cells and in immunocompetent C57BL6 mice targeting both T cells and myeloid cells.

In some embodiments, RAMBO and cisplatin treatment of cancer cells activated anti-tumor immunity to sensitize cancer cells to immunotherapy. In some embodiments, RAMBO and cisplatin treatment increased NK cell activity. In some embodiments, RAMBO and cisplatin treatment sensitizes cancer cells to checkpoint inhibitor therapy, such as, but not limited to anti-PD 1.

### II. Additional Cancer Therapies

In certain embodiments, the compositions and methods of the present embodiments involve a combination therapy of an oncolytic virus (e.g., oHSC, particularly Vstat120-expressing oHSV, such as RAMBO) in combination with at least one DNA damaging agent, such as a platinum-based DNA damaging agent (e.g., cisplatin, oxaliplatin, and/or carboplatin). In some aspects, the subject may be administered the present combination therapy and an additional anti-cancer therapy, such as radiation therapy, surgery (e.g., lumpectomy and a mastectomy), additional chemotherapy, gene therapy, DNA therapy, viral therapy, RNA therapy, immunotherapy, bone marrow transplantation, nanotherapy, monoclonal antibody therapy, or a combination of the foregoing. The additional anti-cancer therapy may be in the form of adjuvant or neoadjuvant therapy. In particular aspects, the additional anti-cancer therapy may be an immune checkpoint inhibitor, such as a PD-1 or CTLA-4 inhibitor.

In some embodiments, the additional anti-cancer therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemo preventative agent. The additional therapy may be one or more of the chemotherapeutic agents known in the art.

The combination therapy (i.e., oncolytic virus and DNA damaging agent) may be administered before, during, after, or in various combinations relative to an additional anti-cancer therapy, such as immune checkpoint therapy. The administrations may be in intervals ranging from concurrently to minutes to days to weeks. In embodiments where the combination therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the combination therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below the combination therapy of an oncolytic virus and DNA damaging agent is “A” and an additional anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wks), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Oncolytic HSV Therapy Modulates Vesicular Trafficking Inducing Cisplatin Sensitivity and Anti-Tumor Immunity

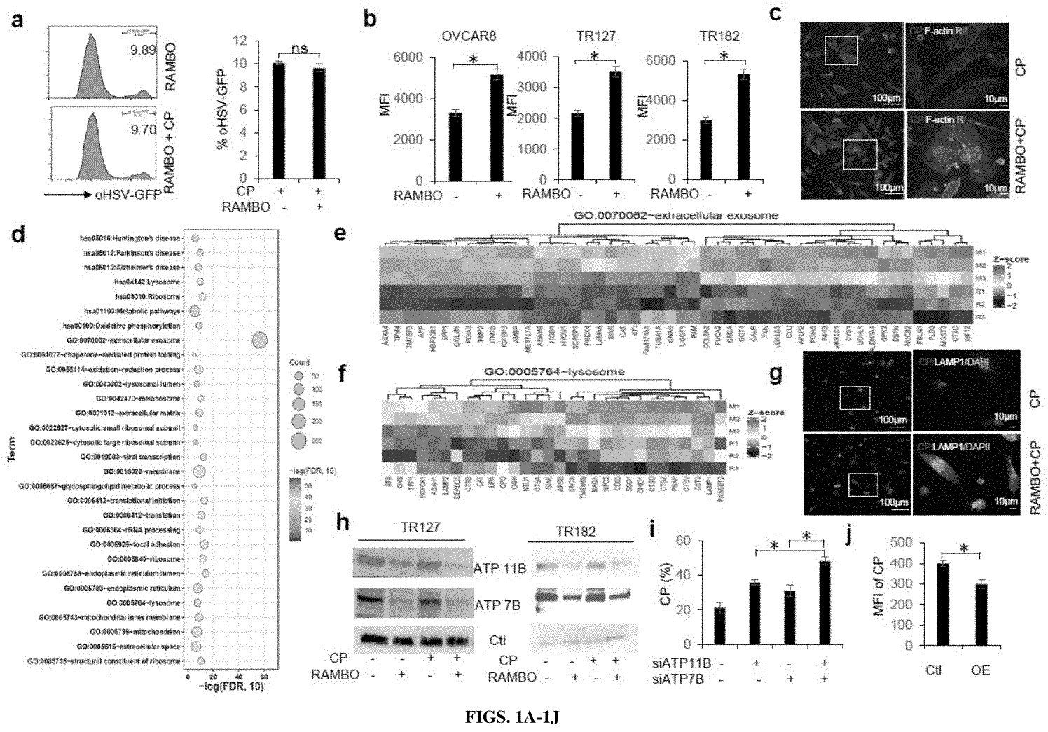

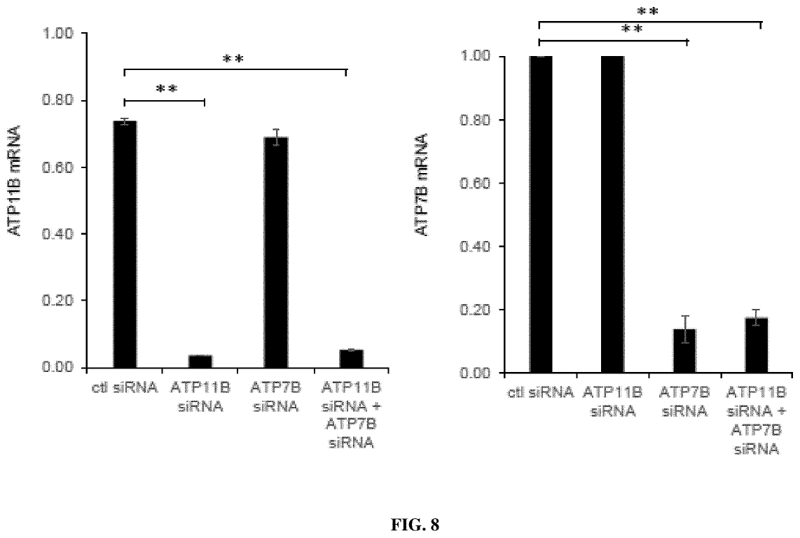

oHSV infection increases cisplatin retention in cisplatin-resistant ovarian cancer cells. To demonstrate the effect of platinum-based chemotherapy (CP) in conjunction with oHSV therapy in OC, the impact of cisplatin (CP) treatment was evaluated on virus replication and also its impact on virus therapy. The effect of CP on virus spread, GFP positive tumor cells were quantified as an indicator of infection and no difference in GFP expression was found in the presence or absence of CP (FIG. 1A). However, when CP accumulation was analyzed following infection with the oHSV RAMBO, a significant increase in Texas-red labeled CP in tumor cells was found by flow cytometry (FIG. 1B). The increased accumulation of CP following RAMBO infection was confirmed by fluorescent confocal microscopy (FIG. 1C). Interestingly, the CP observed inside RAMBO infected cells appeared to be sequestered in cytoplasmic organelles with extra-nuclear punctate localization. To understand the molecular mechanism ascribing to increased CP retention in OC cells treated with RAMBO, gene expression signatures were analyzed between RAMBO infected cells compared to control uninfected cells using RNA sequencing of infected and uninfected OC cells. Gene Ontology (GO) and KEGG pathway analyses of differentially expressed (DE) genes in infected OVTOKO cells indicated a significant global dysregulation of genes in the extracellular exosome related pathway (FIG. 1D). A more detailed heat map analysis to specifically investigate the genes involved in this pathway showed a significant down-regulation of almost all extracellular exosome related genes in cells infected with RAMBO (FIG. 1E). CP localization inside lysosomal compartments, has been shown to be associated with reduced drug efflux and increased cytotoxicity (Safaei et al., 2005; Guerra et al., 2019). Heat map of gene expression changes in the lysosome pathway also indicated a significant reduction in genes associated with this pathway (FIG. 1F), therefore it was investigated if CP was localized to the lysosomal compartments of cells treated with RAMBO. Confocal fluorescent microscopy of un-infected and infected OC cells showed that, indeed, CP co-localized with the lysosomal marker LAMP-1 (FIG. 1G). Collectively, these data indicate that RAMBO infection of OC cells resulted in an alteration of vesicular trafficking, leading to an increased retention of CP. To further characterize this effect, the impact of RAMBO infection was next evaluated on the expression of ATP11B and ATP7B, two vesicular membrane proteins implicated in CP efflux (Moreno-Smith et al., 2013; Vyas et al., 2019). Western blot analysis revealed a significant reduction in the protein expression of both ATP11B and ATP7B in two different OC cells following infection, while the addition of CP had no effect (FIG. 1H). In the absence of viral infection inhibition of both the genes using siRNA resulted in increased CP retention, (FIG. 1I, FIG. 8). Similarly, when both genes were overexpressed there was a significant reduction in CP retention in OC cells (FIG. 1J). Together, these results indicate that expulsion of CP via the exosome/lysosome pathway may be compromised due to OV infection.

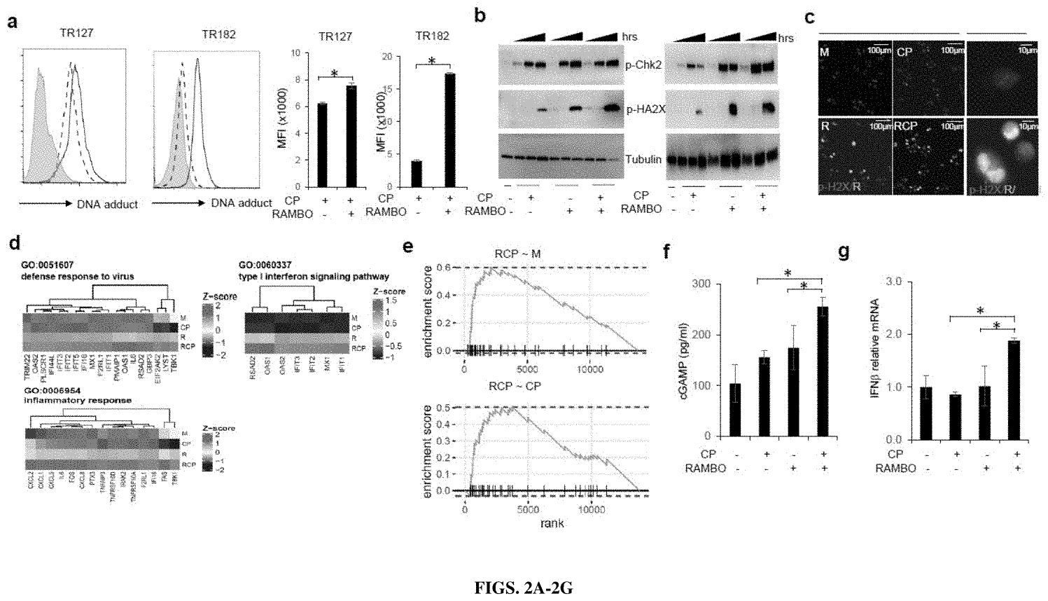

oHSV and CP increase DNA damage and STING mediated immunity. To evaluate if increased CP in OC cells also resulted in increased DNA damage in these cells, the amount of CP-specific DNA adducts was evaluated by flow cytometry. Indeed, compared to uninfected control cells, infection with RAMBO resulted in a significant increase in CP-specific DNA adducts in two different OC cell lines, TR127 and TR182 (FIG. 2A). Consistent with this, western blot analysis of TR127 and TR182 cells infected with RAMBO and/or CP revealed an additive increase of both p-H2AX and p-CHK2 proteins in cells treated with the combination of RAMBO and CP compared to cells treated with either agent alone (FIG. 2B). It has been shown that excessive damaged DNA can leak from the nuclear membrane into the cytosolic space, re-locating in peri-nuclear areas called micronuclei (Mackenzie et al., 2017). This results in the activation of cGAS and STING producing pro-inflammatory cytokines (Harding et al., 2017). Fluorescence microscopy analysis of OC cells treated with CP and RAMBO revealed an increase in the presence of micronuclei in cells treated with combination of CP and RAMBO compared to treatment with either agent alone (FIG. 2C). Gene expression analysis of cells treated with either RAMBO, CP, or the combination showed a significant increase in the following Gene Ontology (GO) pathways: defense response to virus, inflammatory response, and type I interferon signaling pathway (FIG. 2D). Gene set enrichment analysis (GSEA) also revealed a significant enrichment of the STING-IFN pathway in cells treated with the combination of CP and RAMBO (FIG. 2E). Consistent with this, a significant activation of the cGAS/STING pathway was found in cells treated with the combination RAMBO and CP as measured by cGAMP secretion and IFNβ gene expression compared to cells treated with either therapy alone (FIG. 2F, G).

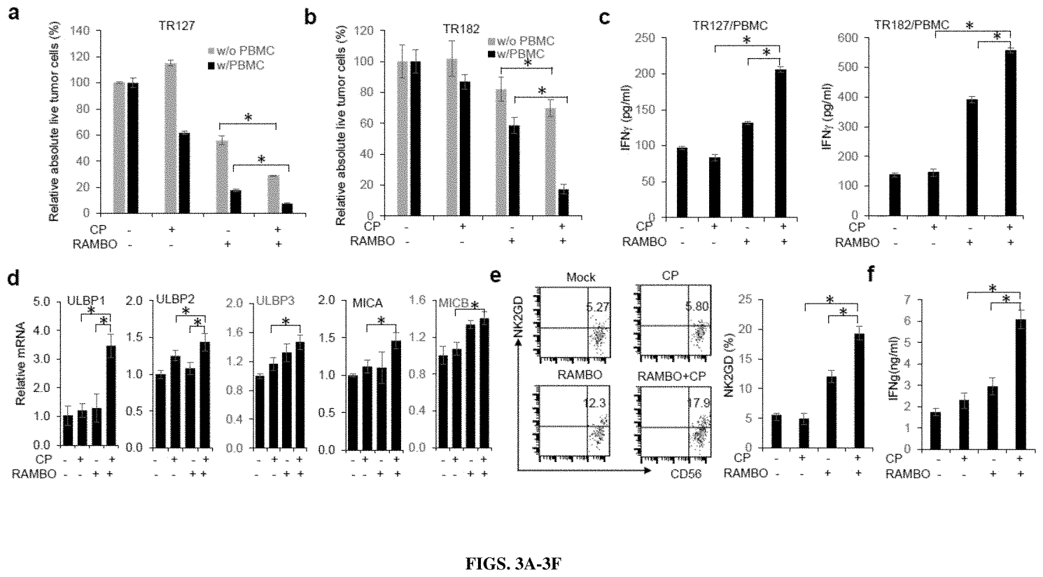

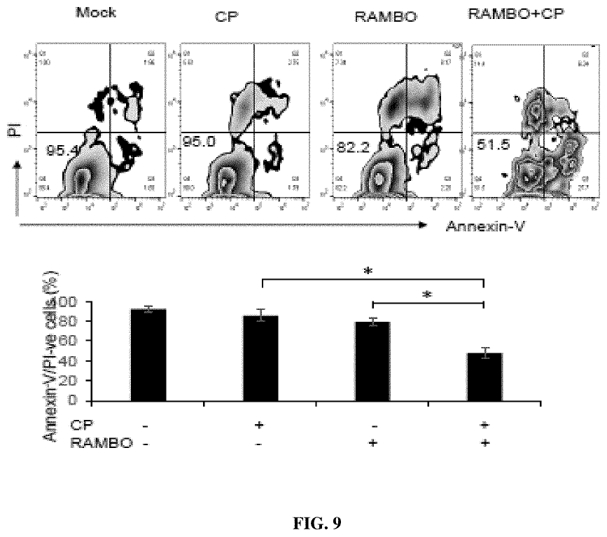

Activation of innate anti-tumor immunity by OC cells treated with RAMBO and CP. Activation of the STING signaling pathway has been shown to result in an anti-tumor immune response. Therefore, the impact of CP and oHSV combination therapy was next evaluated on PBMC-mediated tumor cell killing. Treatment of TR127 and TR182 primary human-derived OC cells with CP and/or RAMBO showed that the combination therapy resulted in an additive increase in tumor cell killing compared to either treatment alone (grey bars in FIG. 3A, B, FIG. 9). When treated cells were overlaid with human donor-derived PBMCs, there was a significant increase in the sensitization of OC cells to PBMC-mediated tumor cell killing when the combination therapy was used compared to either treatment alone (black bars in FIG. 3A, B). Accordingly, PBMCs cultured with tumor cells treated with both CP and RAMBO released a significantly greater amount of IFNγ, indicating increased PBMC activation (FIG. 3C).

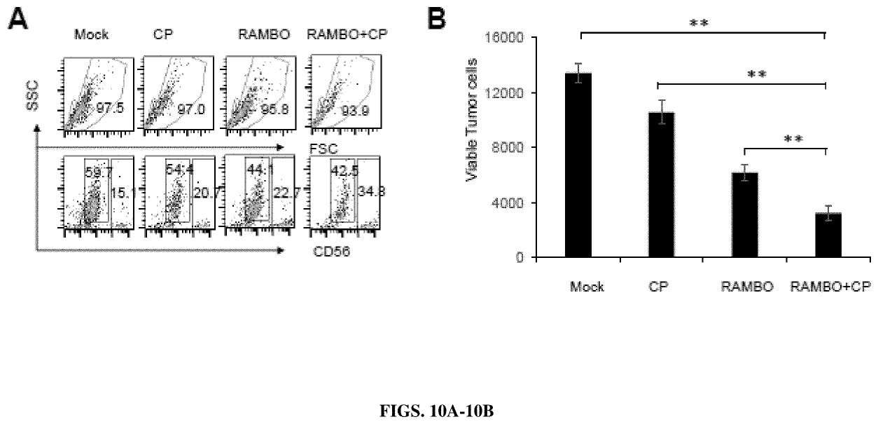

Activation of the STING signaling pathway has been implicated in the induction of NKG2D ligands on tumor cells (Raulet et al., 2017), thus the effect of CP and/or RAMBO treatment on expression of ULBP1, ULBP2, ULBP3, MICA, and MICB was next examined by qRT-PCR. Results showed that OVTOKO cells treated with the combination CP and RAMBO have a significant increase in expression of each NKG2D ligand measured as compared to either treatment alone (FIG. 3D). Co-culturing OVTOKO cells treated with RAMBO and/or CP with human donor-derived NK cells revealed a significant increase in NKG2D expression on CD56⁺ NK cells by flow cytometry and a significant increase in IFNγ secretion, compared NK cells co-cultured with OVTOKO cells treated with either agent alone (FIG. 3E, 3F). Consistent with increased activation flow-cytometry revealed increased tumor cell killing in co-cultures of NK and tumor cells treated with the combination (FIG. 10).

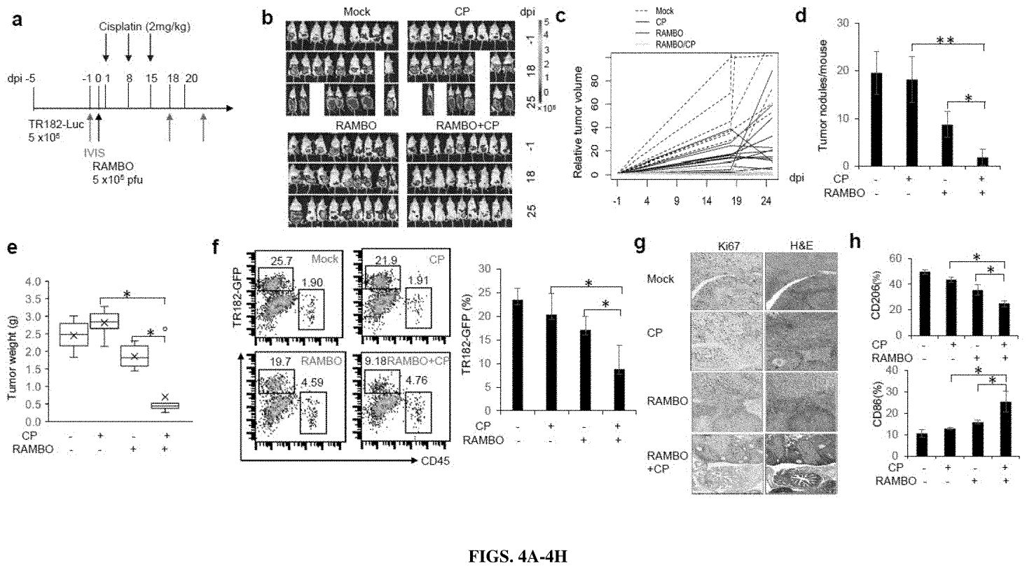

Anti-tumor efficacy of combination CP and RAMBO in a primary human patient derived cisplatin resistant OC in vivo. Next, the impact of combination CP and RAMBO on tumor burden was evaluated in a primary human-derived metastatic OC model in vivo. NSG mice (n=9/group) were implanted with TR182 cells expressing luciferase and GFP (TR182-Luc-GFP) intra-peritoneally. Five days following implant, mice were treated with or without a single injection of RAMBO and administered CP or saline control starting one day later, once a week for three weeks (FIG. 4A). IVIS imaging for tumor-encoded luciferase revealed a significant reduction in tumor burden of mice treated with both RAMBO and CP compared to mice treated with either agent alone (FIG. 4B, 4C). All mice were sacrificed on day 28 post-treatment and the number of tumor nodules were harvested and quantified. Mice treated with the combination therapy showed a significant reduction in the number of tumor nodules and tumor weight compared to mice treated with either agent alone (FIG. 4D, 4E). Flow cytometry analysis of GFP tumor cells in these mice also showed a significant reduction in percentage of tumor cells in the remaining tumor nodules (FIG. 4F). Immuno-histochemical analysis of tumor sections by H&E and Ki67 staining also showed a reduction in proliferating tumor cells in mice treated with both drugs compared to mice treated with either drug alone (FIG. 4G). Immuno-phenotyping of tumor nodules by flow cytometry revealed a significant decrease in % CD206⁺ M2 macrophages and a significant concurrent increase in % CD86⁺ M1 macrophages (gated on the CD45⁺ F4/80⁺ cell population) from mice treated with the combination therapy compared to either treatment alone (FIG. 4H). Collectively, these results reveal an overall reduction in immunosuppression with the combination therapy compared to either therapy alone.

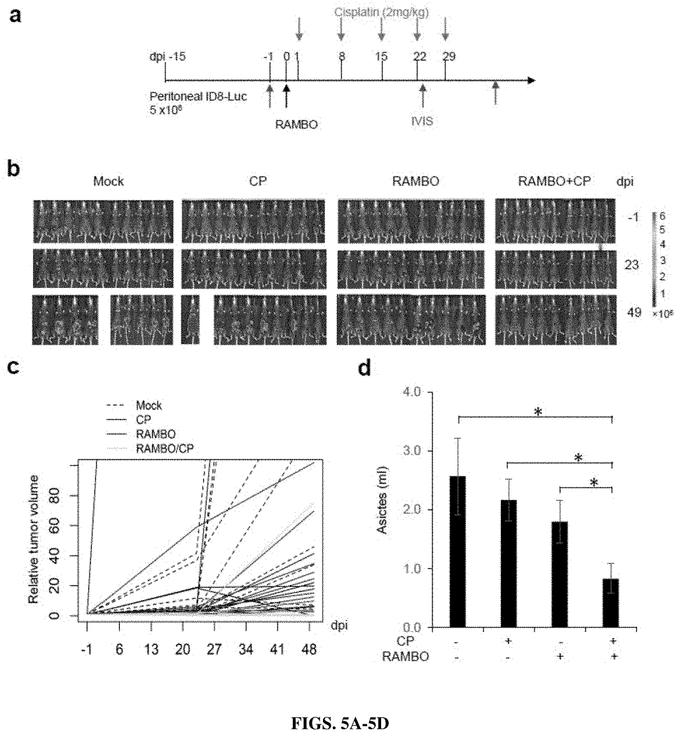

Induction of anti-tumor immunity by treatment with RAMBO and CP. The impact of combination CP and RAMBO on tumor burden was next evaluated in a syngeneic ovarian cancer mouse model. C57BL/6 mice were implanted with ID8-luciferase (ID8-Luc) cells intraperitoneally to develop metastatic OC. Fifteen days post-implantation, tumor bearing animals were treated with or without a single injection of RAMBO and then treated with CP or saline control once a week for five weeks (FIG. 5A). IVIS imaging revealed a significant reduction in tumor growth of mice treated with the combination of RAMBO and CP compared to either treatment alone (FIG. 5B, 5C). Consistent with this, there was significantly less ascites fluid in the abdomen of these mice (FIG. 5D).

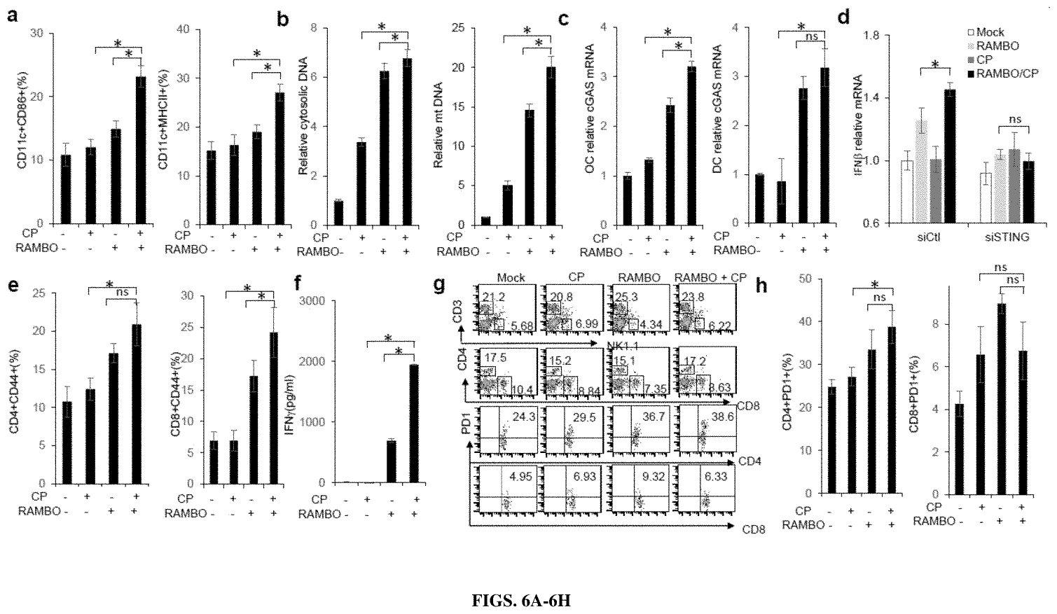

To evaluate the role played by anti-tumor immunity in this model, splenocytes were analyzed in mice treated with either the single agent or the combination three days following RAMBO treatment (three days following RAMBO) by flow cytometry. Results show an increased percentage of CD86⁺ CD11c⁺ and MHCII⁺CD11c⁺ dendritic cells (DCs) in mice treated with the combination of CP and RAMBO compared to either treatment alone (FIG. 6A). To examine the effect of the combination therapy on DC activation in vitro, OVTOKO treated with RAMBO, CP, or both were co-cultured with human donor PBMC-derived DCs in a modified trans-well chamber. 48 hours later DCs were harvested and cytosolic and total DNA was quantified. Results showed that DCs co-cultured with OVTOKO cells treated with the combination therapy had a significant increase in genomic and mitochondrial DNA content in their cytoplasm relative to DCs co-cultured with OVTOKO cells treated with either single agent alone (FIG. 6B). Consistent with increased DNA uptake, a significant increase in cGAS gene expression was also found in both OC (FIG. 6C, left panel) and DCs cells (FIG. 6C, right panel) when OC cells were treated with the combination therapy relative to either treatment alone. Importantly, DCs co-cultured with OCs treated with the combination therapy elicited a robust production of IFNβ, and this was abrogated when DCs were knocked down for STING (FIG. 6D). Together these results implicate a significant role for STING signaling in the DC response to OC cells treated with combination CP and RAMBO.

It was next sought to evaluate the impact of DC activation via OC treatment with combination CP and RAMBO on T cell activation. OVTOKO cells were treated with RAMBO and/or CP and co-cultured with PBMC-derived DCs and PBMC-derived CD3⁺ T cells for 96 hrs. T cell activation was measured as % CD44⁺CD4⁺ and % CD44⁺CD8⁺ by flow cytometry (Schumann et al., 2015). Results showed a significant increase in % CD44⁺CD4⁺ and % CD44⁺CD8⁺ when these cells were co-cultured with OC cells treated with the combination therapy, compared to T cell co-cultured with OC cells treated with either agent alone (FIG. 6E). Along these same lines, a significant increase was measured in IFNγ secretion in the co-culture supernatants when T cells and DCs were co-cultured with OC cells treated with both CP and RAMBO compared to either agent alone (FIG. 6F). Next the effect of combination of CP and RAMBO was evaluated on T cell activation in vivo. Interestingly, despite the increased DC activation observed in the in vitro co-culture system, there was no notable change in cell surface PD1 expression on T cells from mice treated with both CP and RAMBO relative to RAMBO treatment alone (FIG. 6G, H). These results indicate that while RAMBO and CP combination has the potential to induce anti-tumor immunity, additional factors are limiting the activation of effector T cells.

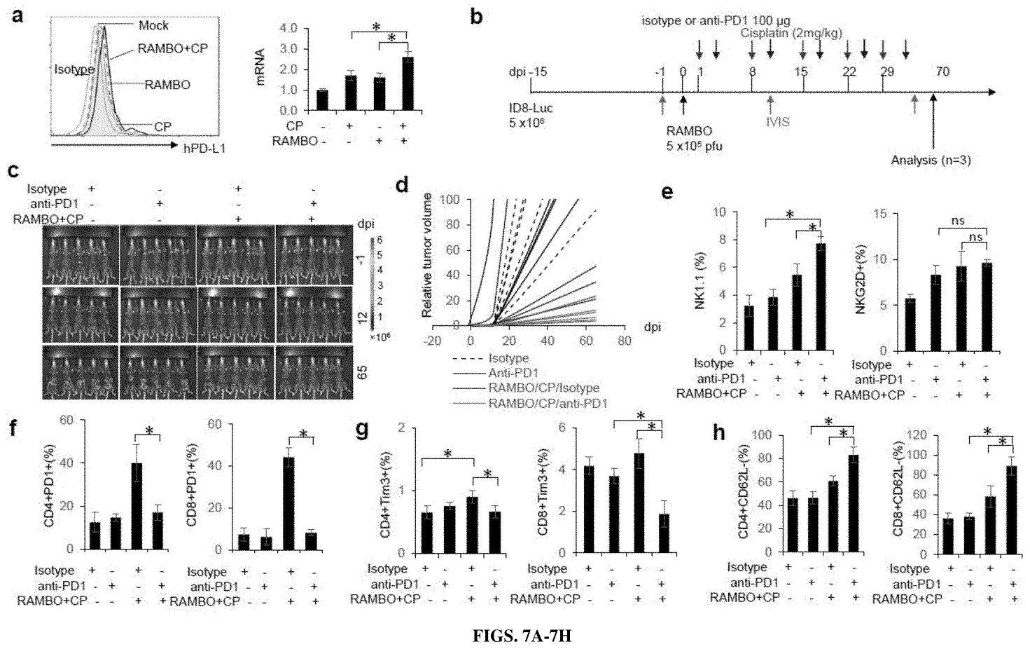

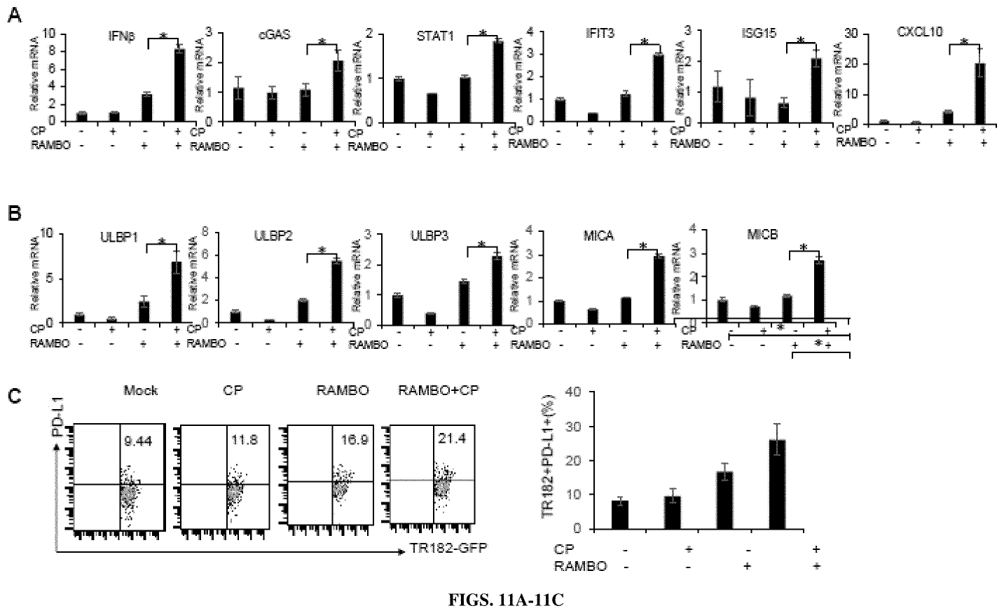

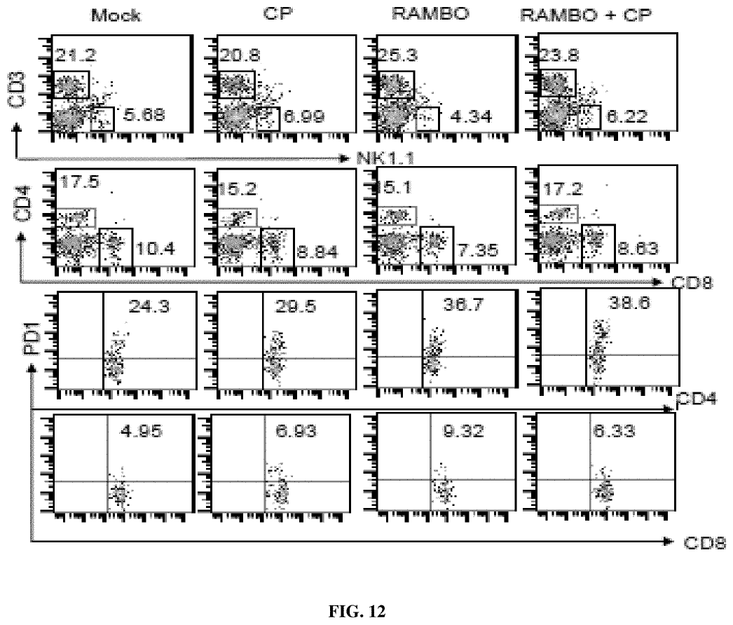

CP and RAMBO treatment sensitizes tumors to checkpoint inhibition: It has been shown that PD-L1 expression on tumor cells suppresses T cell signaling, therefore the impact of combination therapy was next evaluated on PD-L1 expression on OC cells in vitro and in vivo. Indeed, when we treated TR127 and OVTOKO cells with CP and/or RAMBO in vitro there was a significant increase in PD-L1 expression as measured by flow cytometry and qRT-PCR, respectively, compared to either treatment alone (FIG. 7A). Analysis of TR182-Luc-GFP cells harvested 28 days following RAMBO infection from the in vivo model described in FIG. 4, also showed a significant increase in PD-L1 expression on tumor cells from mice treated with the combination therapy, compared to mice treated with either therapy alone (FIG. 11). As expression of PD-L1 on tumor cells has been shown to be a prognostic marker for response to anti-PD-1/anti-PD-L1 therapy (Reck et al., 2016), it was next evaluated if the combination treatment of CP and RAMBO may set the stage to sensitize OC to anti-PD-1/anti-PD-L1 checkpoint inhibition. C57BL/6 mice were implanted with ID8-Luc cells intraperitoneally and then treated with a single injection of RAMBO fifteen days post tumor cell implant. Mice were then treated with CP and either an anti-PD-1 antibody or an isotype control antibody (BioXcell, 100 μg/mouse) once a week for five weeks (FIG. 7B). IVIS imaging revealed a significant reduction in tumor burden in mice treated with RAMBO and CP in the presence of an anti-PD-1 antibody compared to the combination of RAMBO and CP alone, with mice treated with the anti-PD-1 therapy alone showing a minimal slowing of tumor growth (FIG. 7C, 7D). Analysis of splenocytes by flow cytometry, 35 days following RAMBO injection, showed a significant increase in the percentage of NK1.1⁺ cells and a significant reduction in CD4⁺FoxP3⁺ T regulatory cells (L_{egs}) when mice were treated with RAMBO and CP plus the anti-PD-1 antibody compared to RAMBO and CP or anti-PD-1 antibody alone (FIG. 7E, and FIG. 12). Although a change in total T cell number was not observed, mice treated with RAMBO and CP plus anti-PD-1 antibody showed a reduction in virus induced PD-1 expression as measured by % PD-1⁺CD8⁺ and % PD-1⁺CD4⁺ splenic T cells, relative to mice treated with RAMBO and CP alone (FIG. 7F). Additionally, mice treated with the combination of RAMBO and CP plus anti-PD-1 antibody had a reduction in % Tim3⁺CD8⁺ and % Tim3⁺CD4⁺ T cell and an increase in % CD8⁺CD62L⁻ and % CD4⁺CD62L⁻ T cells (FIG. 7G, 7H), indicating an increased activation state of these T cells.

The present studies investigated the impact of combining CP and oHSV for platin resistant OC, and showed that while CP treatment of resistant OC cells did not affect virus replication, viral infection significantly increased intracellular CP retention. RNA sequencing and GO pathway analysis of infected OC cells uncovered a significant dysregulation of the extracellular vesicles (EV)/lysosome pathways in infected cells. It was also shown for the first time that HSV-1 reduced the expression of ATP11B and ATP7B. Thus, the present studies showed increased CP retention in cancer cells after oncolytic HSV therapy. The increased CP retention and ensuing increased DNA damage by oHSV can be used to harness anti-tumor immunity, such as in combination with checkpoint blockade.

### Example 2—Materials and Methods

Cell lines, oncolytic virus and cisplatin reagents. Primary patient derived de-identified cisplatin-resistant ovarian cancer cells TR127 and TR182 were provided by Dr. Gil Mor from Yale University. OVTOKO cells were obtained from ATCC. All ovarian cancer cells were maintained in RPMI medium with 10% FBS. Oncolytic virus RAMBO was constructed in the lab as previously described (Hardcastle et al., 2010). Cisplatin was bought from Selleck Chemicals (#S1166, Houston, Tex.). Texas-red labeled cisplatin was bought from Ursa BioScience (#160175, Bel Air, USA). IFNγ and TNFα released into conditioned medium was analyzed by ELISA. In all in vitro experiments, cells were first infected with RAMBO and, after one hour, virus was washed out and cisplatin was added to culture medium.

Human PBMCs, T cells, NK cells and PBMC-derived dendritic cells. Human PBMCs were isolated from healthy donors using a buffy coat (obtained from Gulf Coast Regional Blood Center, Houston, Tex.) by Ficoll gradient centrifugation. T cells were isolated from PBMCs by negative selection using a T cell isolation kit (#17951, Stemcell Technologies, CA). NK cells were isolated from human PBMCs by negative selection using an NK cell isolation kit (#19665, Stemcell Technologies, CA). Dendritic cells were derived from PBMCs by culturing with 20 ng/ml hGM-CSF (#300-03, PeproTech, USA) and 20 ng/ml hIL-4 (#200-04, PeproTech, USA) for 7 days.

In vitro co-culture assays. Primary ovarian cancer cells were co-cultured with human donor PBMCs, PBMC-derived T cells, PBMC-derived NK cells or PBMC-derived dendritic cells at different ratios for 2-5 days. Tumor cells lysis was analyzed by flow cytometry using Annexin-V/PI gating on cell-tracker labelled, CD45⁻ tumor cells, and normalized with counting microbeads. IFNγ and TNFα secreted from T cells or NK cells were analyzed by ELISA.

Flow cytometry analysis. For cell surface staining, cells were washed with PBS and blocked with Fc blocker (BD Biosciences, San Jose, Calif.). Fluorochrome labeled antibodies (Annexin-V, CD45, CD11c, CD4, CD8, CD11b, Ly6G, Ly6C, PD-1, PD-L1, F4/80, CD56, CD86, HLA-DR, CD206, CD44) were obtained from BD Biosciences (Franklin Lakes, USA), added and stained for 30 min as described (Yoo et al., 2019). For intracellular staining, cells were permeabilized with Fix/Perm buffer (#FC009, R&D systems) for 20 min and then washed with Perm/Wash buffer (R&D systems, Minneapolis, Minn.). Fluorochrome labeled antibody Foxp3 (#560408, BD Biosciences) were diluted in Perm/wash buffer and stained for 30 min as described (Russell et al., 2018). All samples were analyzed on a CytoFlex flow cytometer (Beckman Coulter, CA).

Cytosolic DNA and mitochondrial DNA analysis. Cytosolic DNA and mitochondrial DNA from PBMC-derived dendritic cells co-cultured with ovarian cancer cells were prepared as described (Xu et al., 2017), with modifications. In brief, dendritic cells were cultured in the top chamber of a modified transwell with ovarian cancer cells treated with RAMBO and/or cisplatin in the bottom chamber. Dendritic cells were harvested and purified with CD1a microbeads (#130-051-001, Miltenyi Biotec, USA) and 1×10⁶ dendritic cells were divided into two parts with equal cell numbers (5×10⁵). One part was used to prepare total DNA as input. Another part was used to prepare cytosolic DNA by treating the cells with cytosolic extract buffer containing 150 mM NaCl, 50 mM HEPES and 25 μg/ml digiton. Cytosolic DNA and mitochondrial DNA were quantified by qRT-PCR analysis of POLG expression (cytosolic DNA) and MT-ND1 expression (mitochondrial DNA). Primer sequences for PLOG and MT-ND1 genes are listed in Table 1.

TABLE 1
Primers

| | | SEQ | | SEQ |
|---|---|---|---|---|
| Gene | Forward | ID | Reverse | ID |
| name | Primer sequence (5′-3′) | NO: | Primer sequence (5′-3′) | NO: |
| Human | CCTTAAAGGGCAACTGCTTG | 1 | TCTTGGCTCCAGGATGAAGT | 2 |
| ULBP1 | | | | |
| Human | TGACATCACCGTCATCCCTA | 3 | TTCTCCAGCTGAATGTCACG | 4 |
| ULBP2 | | | | |
| Human | AGCCAGGTGGATCAGAAGAA | 5 | TCAGTGTCAGCCAGTTCCAG | 6 |
| ULBP3 | | | | |
| Human | ATAACCTCACGGTGCTGTCC | 7 | TTTCCGTTCCCTGTCAAGTC | 8 |
| MICA | | | | |
| Human | TTTCTCGCTGAGGGACATCT | 9 | GAATGCAAGCCTCCTTTCTG | 10 |
| MICB | | | | |
| Human | AAACATTCGGGTGGGTGATA | 11 | TGCCACATGTGTCTTCAGGT | 12 |
| ATP11B | | | | |
| Human | GGTCACCCTCCAACTGAGAA | 13 | TGATCTGTCCCACTCCCTTC | 14 |
| ATP7B | | | | |
| Human | ATGAGCAGTCTGCACCTGAAA | 15 | TGAAGCAATTGTCCAGTCCCA | 16 |
| IFN□ | | | | |
| Human | GCTGCACGAGCAAATCTTC | 17 | AAGTGCTGGTCCAGGTTGTC | 18 |
| POLG1 | | | | |
| Human | ACTACAACCCTTCGCTGACG | 19 | GCCTAGGTTGAGGTTGACCA | 20 |
| ND1 | | | | |
| Human | TTCACGTATGTACCCAGAACCC | 21 | TGTCCTGAGGCACTGAAGAAAG | 22 |
| cGAS | | | | |
| Human | CGAAGTCATCTGGACAAGCA | 23 | ATTTGGAGGATGTGCCAGAG | 24 |
| PD-L1 | | | | |
| Human | TCCTGCACCAC- | 25 | TGGTCATGAGTCCTTCCACGATAC | 26 |
| GAPDH | CAACTGCTTAG | | | |

qRT-PCR analysis. Quantitative Real Time-PCR was performed on an ABI Quest studio 3.0 system using Sybr Green (#208054, Qiagen). Gene specific primers (primer sequences listed in table 1) were synthesized from IDT Company (Coralville, Iowa).

In vivo ovarian cancer NSG model. To establish a human metastatic ovarian cancer model, 5×10⁵ TR182-Luciferase (TR182-Luc) cells were injected intraperitoneally into 5-6-week old female NSG mice. Five days later, when tumor luciferase was visible under IVIS imaging, tumor-bearing mice were treated by intraperitoneal injections with or without 5×10⁵ pfu RAMBO once. The mice were also administered intraperitoneally 2 mg/kg cisplatin twice every week and saline as control.

In vivo ovarian cancer immunocompetent mouse model. A murine metastatic peritoneal ovarian cancer model was established in C57BL/6 mice by intraperitoneal injection of 5×10⁶ ID8-Luciferase (ID8-Luc) cells. 14 days later, tumor-encoded luciferase was imaged using IVIS imaging, and mice were randomized to be treated intraperitoneally with 5×10⁵ pfu RAMBO and 2 mg/kg cisplatin once every week. Tumor growth was monitored by IVIS imaging. Early and late anti-tumor T cell immune response was monitored day 3 (early) or day 35 (late) after RAMBO treatment by harvesting splenocytes and analyzing for T cells and myeloid cells.

Immunofluorescence staining. Ovarian cancer cells were plated on chamber slides and treated with RAMBO and/or cisplatin. At the indicated time points following treatment, cells were fixed in 4% PFA for 15 min and permeabilized with 0.01% triton-X in PBS for 15 min. The cells were blocked with 2% BSA in PBS for 1 hour prior to incubation with primary antibodies, phalloidin (#8940, Cell Signaling Technology, 1:1000 dilution), LAMP1 (#9091, Cell Signaling Technology, 1:1000 dilution) diluted in 0.1% BSA overnight. The slides were washed three times with PBS-T and secondary antibody was added for 30 min. DAPI and F-actin were stained for cell nuclei and cell structure. The slides were washed and analyzed under confocal or regular fluorescence microscopy.

Western blot. Whole cell lysates were prepared and loaded onto SDS-PAGE. After transfer, the membrane was blocked with 5% skim milk for 1 hr and then incubated in diluted primary antibodies ATP11B (#ab05377), ATP7B (#ab124973), tubulin (#ab210797) or GAPDH (#ab181602), obtained from Abcam, Cambridge, USA) overnight. The membrane was washed with TBS-T three times and incubated with an HRP-labeled secondary antibody for 1 hr. The membrane was developed using a Bio-Rad developer system.

Immunohistochemistry. Tumor tissues were fixed and embedded in paraffin and sections were prepared. After de-paraffin and antigen retrieval, the tissue sections were incubated in primary antibody Ki-67 (#ab15580, Abcam, USA) overnight. After washing with PBS-T three times, the sections were incubated in secondary antibody for 1 hr. The signal was developed using an ABC kit from Vector Laboratories (#PK-7200, Burlingame, USA).

RNA library construction and data analysis. For RNA sequencing (RNAseq), total RNA was prepared from OVTOKO cells treated with RAMBO (MOI=1) and/or cisplatin (5 μM) for 16 hours. Total RNA was extracted using RNeasy mini kit (#74104, Qiagen, Germany). Poly (A)-tailed messenger RNA was enriched and the RNAseq library was constructed by the UTHealth Cancer Genomics Core following the manufacturer's instructions for the KAPA mRNA HyperPrep Kit (#KK8581, Roche Holding AG, Switzerland) and the KAPA Unique Dual-indexed Adapter kit (KK8727, Roche Holding AG, Switzerland). RNAseq data was generated by an Illumina Nextseq 550 using the 75 bp pair-ended running mode.

Raw mRNA sequence reads were pre-processed using Cutadapt (v1.15) to remove bases with quality scores<20 and adapter sequences (Martin, 2011). Clean RNAseq reads were aligned to the reference genome GRCh38.83 with STAR (v2.5.3a) (Dobin et al., 2013). Gene abundance was measured by HTseq-count uniquely-mapped read number with default parameters and using annotations from ENSEMBL v83. Only the genes with >5 reads in at least one sample were used for differential expression analysis by DESeq2 software (Ander and Huber, 2010), which implements a negative binomial distribution model. Resulting p values were adjusted using the Benjamini and Hochberg's approach (Benjamini and Hochberg, 1995) to control for false discovery rate (FDR). Genes with FDR<0.05 were considered as differentially expressed for follow up analysis. Gene set enrichment analysis (GSEA) was conducted using RDAVID WebService (v1.19.0) (Fresno and Fernandez, 2013) for Gene Ontology (GO) terms and R package fgsea for KEGG pathway analysis, respectively. The enrichment p values were adjusted by following Benjamini and Hochberg's approach (Benjamini and Hochberg, 1995).

Statistical analysis. All quantitative results are displayed as the mean±s.d. (standard deviation). The statistical difference between two groups was compared using a Mann-Whitney u test or a Student's t test. If more than 2 groups were compared, ANOVA was used. Statistical analysis was determined using Prism5 software (GraphPad Software, Inc., La Jolla, Calif.). A p value of less than 0.05 was considered statistically significant.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Adusumilli, P. S., et al., Cisplatin-induced GADD34 upregulation potentiates oncolytic viral therapy in the treatment of malignant pleural mesothelioma. Cancer Biol Ther, 2006. 5(1): p. 48-53.
Agarwal, R. and S.B. Kaye, Ovarian cancer: strategies for overcoming resistance to chemotherapy. Nat Rev Cancer, 2003. 3(7): p. 502-16.
Anders, S. & Huber, W. Differential expression analysis for sequence count data. Genome Biol 11, R106, doi:10.1186/gb-2010-11-10-r106 (2010).
Austin-Ward and Villaseca, Revista Medica de Chile, 126(7):838-845, 1998.
Benjamini, Y. & Hochberg, Y. Controlling the False Discovery Rate: A Practical and Powerful Approach to Multiple Testing. Journal of the Royal Statistical Society. Series B (Methodological) 57, 289-300 (1995).
Bukowski et al., Clinical Cancer Res., 4(10):2337-2347, 1998.
Chan, J. K., et al., Do clear cell ovarian carcinomas have poorer prognosis compared to other epithelial cell types? A study of 1411 clear cell ovarian cancers. Gynecol Oncol, 2008. 109(3): p. 370-6.
Christodoulides et al., Microbiology, 144(Pt 11):3027-3037, 1998.
Davidson et al., J. Immunother., 21(5):389-398, 1998.
Davis, A., A. V. Tinker, and M. Friedlander, “Platinum resistant” ovarian cancer: what is it, who to treat and how to measure benefit? Gynecol Oncol, 2014. 133(3): p. 624-31.
Deng, L., et al., STING-Dependent Cytosolic DNA Sensing Promotes Radiation-Induced Type I Interferon-Dependent Antitumor Immunity in Immunogenic Tumors. Immunity, 2014. 41(5): p. 843-52.
Dobin, A. et al. STAR: ultrafast universal RNA-seq aligner. Bioinformatics 29, 15-21, doi:10.1093/bioinformatics/bts635 (2013).
Fresno, C. & Fernandez, E. A. RDAVIDWebService: a versatile R interface to DAVID. Bioinformatics 29, 2810-2811 (2013).
Guerra, F. et al. Modulation of RAB7A Protein Expression Determines Resistance to Cisplatin through Late Endocytic Pathway Impairment and Extracellular Vesicular Secretion. Cancers (Basel) 11, doi:10.3390/cancers11010052 (2019).
Hanibuchi et al., Int. J. Cancer, 78(4):480-485, 1998.
Hardcastle, J. et al. Enhanced antitumor efficacy of vasculostatin (Vstat120) expressing oncolytic HSV-1. Molecular therapy: the journal of the American Society of Gene Therapy 18, 285-294, doi:10.1038/mt.2009.232 (2010).
Harding, S. M. et al. Mitotic progression following DNA damage enables pattern recognition within micronuclei. Nature 548, 466-470, doi:10.1038/nature23470 (2017).
Hellstrand et al., Acta Oncologica, 37(4):347-353, 1998.
Holmes, D., Ovarian cancer: beyond resistance. Nature, 2015. 527(7579): p. 5217.
Hui and Hashimoto, Infection Immun., 66(11):5329-5336, 1998.
International Patent Publication No. WO2006/121168
International Patent Publication No. WO2015016718
Kasuya, H., et al., Suitability of a US3-inactivated HSV mutant (L1BR1) as an oncolytic virus for pancreatic cancer therapy. Cancer Gene Ther, 2007. 14(6): p. 533-42.
Li, T. and Z. J. Chen, The cGAS-cGAMP-STING pathway connects DNA damage to inflammation, senescence, and cancer. J Exp Med, 2018. 215(5): p. 1287-1299.
Mackenzie, K. J. et al. cGAS surveillance of micronuclei links genome instability to innate immunity. Nature 548, 461-465, doi:10.1038/nature23449 (2017).
Martin, M. Cutadapt removes adapter sequences from high-throughput sequencing reads. EMBnet.journal 17, 10-12, doi:10.14806/ej.17.1.200 (2011).
Moreno-Smith, M. et al. ATP11B mediates platinum resistance in ovarian cancer. J Clin Invest 123, 2119-2130, doi:10.1172/JCI65425 (2013).
Pardoll, Nat Rev Cancer, 12(4): 252-64, 2012.
Qin et al., Proc. Natl. Acad. Sci. USA, 95(24):14411-14416, 1998.
Raulet, D. H., Marcus, A. & Coscoy, L. Dysregulated cellular functions and cell stress pathways provide critical cues for activating and targeting natural killer cells to transformed and infected cells. Immunol Rev 280, 93-101, doi:10.1111/imr.12600 (2017).
Reck, M. et al. Pembrolizumab versus Chemotherapy for PD-L1-Positive Non-Small-Cell Lung Cancer. N Engl J Med 375, 1823-1833, doi:10.1056/NEJMoa1606774 (2016).
Russell, L. et al. PTEN expression by an oncolytic herpesvirus directs T-cell mediated tumor clearance. Nat Commun 9, 5006, doi:10.1038/s41467-018-07344-1 (2018).
Safaei, R. et al. Intracellular localization and trafficking of fluorescein-labeled cisplatin in human ovarian carcinoma cells. Clin Cancer Res 11, 756-767 (2005).
Schumann, J., Stanko, K., Schliesser, U., Appelt, C. & Sawitzki, B. Differences in CD44 Surface Expression Levels and Function Discriminates IL-17 and IFN-gamma Producing Helper T Cells. PLoS One 10, e0132479, doi:10.1371/journal.pone.0132479 (2015).
SEER Stat Fact Sheets: Ovary Cancer. 2015.
Simpson, G. R., et al., Combination of a fusogenic glycoprotein, pro-drug activation and oncolytic HSV as an intravesical therapy for superficial bladder cancer. Br J Cancer, 2012. 106(3): p. 496-507.
Snyder, M. B., et al., The in-vitro and in-vivo efficacy of cisplatin and analogues in the treatment of herpes simplex virus-II infections. J Antimicrob Chemother, 1987. 19(6): p. 815-22.
U.S. Pat. No. 4,870,287
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,846,945
U.S. Pat. No. 8,008,449
U.S. Pat. No. 8,354,509
U.S. Pat. No. 8,450,106
U.S. Pat. No. 8,735,553
U.S. Patent Publication No. US20110008369
U.S. Patent Publication No. US2014022021
U.S. Patent Publication No. US20140294898
Vyas, A., Duvvuri, U. & Kiselyov, K. Copper-dependent ATP7B up-regulation drives the resistance of TMEM16A-overexpressing head-and-neck cancer models to platinum toxicity. Biochem J 476, 3705-3719, doi:10.1042/BCJ20190591 (2019).
Xu, M. M. et al. Dendritic Cells but Not Macrophages Sense Tumor Mitochondrial DNA for Cross-priming through Signal Regulatory Protein alpha Signaling. Immunity 47, 363-373 e365, doi:10.1016/j.immuni.2017.07.016 (2017).
Xu, M. M., et al., Dendritic Cells but Not Macrophages Sense Tumor Mitochondrial DNA for Cross-priming through Signal Regulatory Protein alpha Signaling. Immunity, 2017. 47(2): p. 363-373 e5.
Yoo, J. Y. et al. oHSV therapy increases trametinib access to brain tumors and sensitizes them in vivo. Neuro Oncol, doi:10.1093/neuonc/noz079 (2019).
WO2009/114335
WO2009/101611
WO2010/027827 and WO2011/066342

U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304
International Patent Application No. WO2001014424, WO2000037504, and U.S. Pat. No. 8,017,114
WO 01/14424
U.S. Pat. Nos. 5,844,905, 5,885,796 and International Patent Application Nos. WO1995001994 and WO1998042752
U.S. Pat. No. 8,329,867

1. A method of treating platinum-resistant ovarian cancer in a subject comprising administering an effective amount of an oncolytic herpes simplex virus (oHSV) and a platinum-based chemotherapy to the subject.

2. (canceled)

3. (canceled)

4. The method of claim 1, wherein the platinum-based chemotherapy comprises cisplatin, oxaliplatin, and/or carboplatin.

5. The method of claim 4, wherein the platinum-based chemotherapy comprises cisplatin.

6. (canceled)

7. The method of claim 1, wherein the oHSV is an HSV-1 strain.

8. The method of claim 6, wherein the oHSV is a Vstat120-expressing (RAMBO) oHSV.

9. The method of claim 1, wherein the amount of the oHSV is effective to stimulate an innate and/or adaptive immune response in the subject.

10. The method of claim 1, wherein the amount of the oHSV is effective to stimulate an anti-tumor T-cell response in the subject.

11. The method of claim 1, wherein the amount of the oHSV is effective to stimulate PD1-PDL1 signaling in T-cells and/or myeloid cells of the subject.

12. The method of claim 1, wherein the oHSV is administered after the platinum-based chemotherapy.

13. The method of claim 1, wherein the oHSV is administered before or essentially simultaneously with the platinum-based chemotherapy.

14. The method of claim 1, wherein the oHSV is administered within 1 day of the platinum-based chemotherapy.

15. The method of claim 1, wherein the oHSV is administered within 3 hours of the platinum-based chemotherapy.

16. The method of claim 1, wherein the oHSV is administered to the subject 2, 3, 4 or 5 times.

17-19. (canceled)

20. The method of claim 1, wherein the platinum-resistant ovarian cancer is metastatic platinum-resistant ovarian cancer.

21-23. (canceled)

24. The method of claim 1, wherein the platinum-resistant ovarian cancer is a cisplatin resistant ovarian cancer.

25-29. (canceled)

30. The method of claim 1, further comprising administering a further anti-cancer therapy.

31. (canceled)

32. The method of claim 30, wherein the anti-cancer therapy comprises at least one immune checkpoint inhibitor.

33. The method of claim 32, wherein the immune checkpoint inhibitor inhibits the PD1-PDL1 pathway.

34. The method of claim 32, wherein the immune checkpoint inhibitor is an anti-PD1 or anti-CTLA-4 monoclonal antibody.

35. (canceled)

36. A pharmaceutical composition comprising an oncolytic herpes simplex virus (oHSV), an immune checkpoint inhibitor, and a platinum-based chemotherapeutic agent.

37-40. (canceled)


SEQUENCE LISTING (26 sequences)

SEQ ID NO: 1 - DNA, 20 bp, Artificial sequence
  Synthetic primer
ccttaaagggcaactgcttg

SEQ ID NO: 2 - DNA, 20 bp, Artificial sequence
  Synthetic primer
tcttggctccaggatgaagt

SEQ ID NO: 3 - DNA, 20 bp, Artificial sequence
  Synthetic primer
tgacatcaccgtcatcccta

SEQ ID NO: 4 - DNA, 20 bp, Artificial sequence
  Synthetic primer
ttctccagctgaatgtcacg

SEQ ID NO: 5 - DNA, 20 bp, Artificial sequence
  Synthetic primer
agccaggtggatcagaagaa

SEQ ID NO: 6 - DNA, 20 bp, Artificial sequence
  Synthetic primer
tcagtgtcagccagttccag

SEQ ID NO: 7 - DNA, 20 bp, Artificial sequence
  Synthetic primer
ataacctcacggtgctgtcc

SEQ ID NO: 8 - DNA, 20 bp, Artificial sequence
  Synthetic primer
tttccgttccctgtcaagtc

SEQ ID NO: 9 - DNA, 20 bp, Artificial sequence
  Synthetic primer
tttctcgctgagggacatct

SEQ ID NO: 10 - DNA, 20 bp, Artificial sequence
  Synthetic primer
gaatgcaagcctcctttctg

SEQ ID NO: 11 - DNA, 20 bp, Artificial sequence
  Synthetic primer
aaacattcgggtgggtgata

SEQ ID NO: 12 - DNA, 20 bp, Artificial sequence
  Synthetic primer
tgccacatgtgtcttcaggt

SEQ ID NO: 13 - DNA, 20 bp, Artificial sequence
  Synthetic primer
ggtcaccctccaactgagaa

SEQ ID NO: 14 - DNA, 20 bp, Artificial sequence
  Synthetic primer
tgatctgtcccactcccttc

SEQ ID NO: 15 - DNA, 21 bp, Artificial sequence
  Synthetic primer
atgagcagtctgcacctgaaa

SEQ ID NO: 16 - DNA, 21 bp, Artificial sequence
  Synthetic primer
tgaagcaattgtccagtccca

SEQ ID NO: 17 - DNA, 19 bp, Artificial sequence
  Synthetic primer
gctgcacgagcaaatcttc

SEQ ID NO: 18 - DNA, 20 bp, Artificial sequence
  Synthetic primer
aagtgctggtccaggttgtc

SEQ ID NO: 19 - DNA, 20 bp, Artificial sequence
  Synthetic primer
actacaacccttcgctgacg

SEQ ID NO: 20 - DNA, 20 bp, Artificial sequence
  Synthetic primer
gcctaggttgaggttgacca

SEQ ID NO: 21 - DNA, 22 bp, Artificial sequence
  Synthetic primer
ttcacgtatgtacccagaaccc

SEQ ID NO: 22 - DNA, 22 bp, Artificial sequence
  Synthetic primer
tgtcctgaggcactgaagaaag

SEQ ID NO: 23 - DNA, 20 bp, Artificial sequence
  Synthetic primer
cgaagtcatctggacaagca

SEQ ID NO: 24 - DNA, 20 bp, Artificial sequence
  Synthetic primer
atttggaggatgtgccagag

SEQ ID NO: 25 - DNA, 22 bp, Artificial sequence
  Synthetic primer
tcctgcaccaccaactgcttag

SEQ ID NO: 26 - DNA, 24 bp, Artificial sequence
  Synthetic primer
tggtcatgagtccttccacgatac